(19) United States
(12) Patent Application Publication
(10) Pub. No.: US 2026/0355259 A1
(43) Pub. Date: Nov. 12, 2026

(54) COMPOSITION FOR PREVENTING AND TREATING ATOPY COMPRISING STICHOPUS JAPONICUS-DERIVED EXTRACELLULAR VESICLES

(71) Applicant: GLOPEX CO., LTD., Hwaseong-si (KR)

(72) Inventors: Sang-Hyug Park, Suwon-si (KR); Sung-Han Jo, Busan (KR); Seon-Hwa Kim, Paju-si (KR)

(73) Assignee: GLOPEX CO., LTD., Hwaseong-si (KR)

(21) Appl. No.: 19/711,828

(22) Filed: Jun. 17, 2026

Related U.S. Application Data
(63) Continuation of application No. PCT/KR2025/000365, filed on Jan. 8, 2025.

(30) Foreign Application Priority Data
Jan. 9, 2024 (KR) 10-2024-0003301

(51) Int. Cl. A61K 35/616; A61P 17/00

(52) U.S. Cl. CPC A61K 35/616; A61P 17/00

(57) ABSTRACT

The present invention relates to a composition for treating atopic dermatitis, the composition comprising Stichopus japonicus-derived extracellular vesicles. Treating LPS-induced inflammation macrophages and mouse models with Stichopus japonicus-derived extracellular vesicles was found to activate anti-inflammatory effects. Accordingly, the composition comprising Stichopus japonicus-derived extracellular vesicles has an excellent therapeutic effect on atopic dermatitis and can be utilized in the field of researching better therapeutic effects, and thus can be used in useful businesses related to atopic dermatitis.

### CROSS-REFERENCE TO RELATED APPLICATION

This application is a continuation of International Application No. PCT/KR2025/000365, filed on Jan. 8, 2025, which claims priority to Korean Patent Application No. 10-2024-0003301, filed on Jan. 9, 2024, the entire disclosures of which are incorporated herein by reference.

### BACKGROUND

### Field

The present invention relates to a composition for preventing and treating atopic dermatitis, comprising Stichopus japonicus-derived extracellular vesicles (EVs).

### Description of Related Art

Atopic dermatitis is a recurrent, chronic inflammatory skin condition characterized by severe pruritus, with symptoms including itching, erythema, edema, exudation, and excoriation, and approximately 10% to 15% of children have atopic dermatitis. The symptoms of atopic dermatitis may be aggravated by environmental antigens and irritants. Although the precise etiology of atopic dermatitis has not been fully elucidated, hyperactivation of nucleotide-binding oligomerization domain (NOD)-like receptors (NLRs), which are specifically activated to cause appropriate immune responses depending on the characteristics of individual infectious pathogens, has been proposed as one of the causative factors. Atopic dermatitis is understood to arise from the interaction of multiple factors, including genetic, environmental, and immunological factors. Due to these characteristics, atopic dermatitis is difficult to effectively ameliorate with treatment targeting only one of the causative factors, and the importance of prevention and management has been emphasized, considering its chronic and relapsing nature. Currently available treatments include wet dressings, oral and injectable steroids, and immunomodulatory agents, but these treatments are often associated with adverse effects.

Conventionally, for the treatment of atopic dermatitis, various agents have been employed, including ingredients such as ceramides, linoleic acid, plant oils, and mineral oils; steroid preparations such as hydrocortisone and substances with enhanced antibacterial and anti-inflammatory properties; DNA synthesis inhibitors via phototherapy; cell hyperproliferation inhibitors; and anti-inflammatory and antipruritic agents.

However, concerns have been raised regarding the adverse effects of steroid preparations, including the suppression of epidermal growth and other adverse effects, urea peroxide may cause excessive skin irritation, and antihistamines and other antibiotics have a high potential for causing side effects such as microbial resistance and photosensitivity. Accordingly, research on natural substances with fewer adverse effects as agents for treating atopic dermatitis has been actively conducted. Representative examples include Artemisia vulgaris extract (Korean Patent Registration No. 10-0377262), extracts of Scutellaria baicalensis and Achyranthes bidentata (Korean Patent Registration No. 10-0451444), and eucalyptus and tea tree oils (Korean Patent Registration No. 10-0597997). Nevertheless, these natural substances still present problems such as insufficient efficacy or hypersensitivity reactions in sensitive skin.

Meanwhile, extracellular vesicles (EVs) are nano-sized vesicular particles secreted by cells to the outside, ranging in size from 30 to 1,000 nm, and are enclosed by a lipid bilayer membrane. EVs are involved in various physiological processes, including intercellular signaling, immune responses, and cell proliferation and differentiation. EVs may be broadly classified into three types: “exosomes,” which are the most common type of EV formed in and secreted from the endoplasmic reticulum, containing various substances including proteins, lipids, RNA, and DNA; “microvesicles,” which are formed by the detachment of portions of the cell membrane and contain similar components to exosomes; and “apoptotic bodies,” which are formed during the process of cell apoptosis and play an important role in immune responses. Although such EVs have been found to be applicable to the treatment of various diseases, including cancer, neurological disorders, immune disorders, and cardiovascular diseases, the use of Stichopus japonicus-derived EVs for treating atopic dermatitis and the specific mechanisms thereof remain unknown.

Accordingly, the present inventors confirmed that Stichopus japonicus-derived EVs possess anti-inflammatory effects and exhibit preventive and therapeutic effects against atopic dermatitis by engaging in the NLR signaling pathway, thereby completing the present invention.

### SUMMARY

One object of the present invention is to provide a pharmaceutical composition for preventing and treating atopic dermatitis, including Stichopus japonicus-derived extracellular vesicles (EVs).

Another object of the present invention is to provide a food composition for preventing and ameliorating atopic dermatitis, including Stichopus japonicus-derived EVs.

Still another object of the present invention is to provide a cosmetic composition for preventing and ameliorating atopic dermatitis, including Stichopus japonicus-derived EVs.

Yet another object of the present invention is to provide a method of treating atopic dermatitis by administering the above-described extract to a non-human animal.

Through research aimed at discovering natural substances for preventing and treating atopic dermatitis, the present inventors confirmed that Stichopus japonicus-derived extracellular vesicles (EVs) engage in the nucleotide-binding oligomerization domain (NOD)-like receptor signaling pathway in macrophages involved in autoimmune diseases, thereby decreasing pro-inflammatory cytokines and atopic dermatitis-associated factors (interferon gamma inducible protein 16 (IFI16); nucleotide-binding domain, leucine-rich-containing family, pyrin domain-containing-3 (NLRP3)), while increasing anti-inflammatory and defense-related factors, thereby demonstrating that these vesicles can be used as a means for solving the above-described problems.

To achieve the above-described objects, the present invention provides a pharmaceutical composition for preventing and treating atopic dermatitis, including Stichopus japonicus-derived EVs.

The present invention also provides a food composition for preventing and ameliorating atopic dermatitis, including Stichopus japonicus-derived EVs.

The present invention also provides a cosmetic composition for preventing and ameliorating atopic dermatitis, including Stichopus japonicus-derived EVs.

The present invention also provides a method of treating atopic dermatitis by administering the above-described extract to a non-human animal.

It has been confirmed that the composition including the Stichopus japonicus-derived extracellular vesicles (EVs) of the present invention induces an anti-inflammatory function, suppresses skin thickness increase, and ameliorates skin lesions, thereby demonstrating a therapeutic effect on atopic dermatitis through the inhibition of the nucleotide-binding oligomerization domain (NOD)-like receptor (NLR) signaling pathway, which is a known cause of atopic dermatitis. Therefore, the composition can be effectively used for the prevention, treatment, or amelioration of atopic dermatitis.

A pharmaceutical composition for preventing and treating atopic dermatitis according to one embodiment of the present invention may include a Stichopus japonicus-derived EV as an active ingredient.

In one embodiment, the Stichopus japonicus-derived EV may be obtained by enzymatically treating an extracellular matrix (ECM) obtained by decellularizing Stichopus japonicus tissue.

In one embodiment, the enzyme may be collagenase.

In one embodiment, the Stichopus japonicus-derived EV may include an exosome.

In one embodiment, the Stichopus japonicus-derived EV may suppress an inflammatory response by participating in a nucleotide-binding oligomerization domain (NOD)-like receptor signaling pathway in macrophages in which inflammation has been induced by lipopolysaccharide (LPS).

In one embodiment, the Stichopus japonicus-derived EV may decrease the expression of interferon gamma inducible protein 16 (IFI16), a gene associated with the NOD-like receptor signaling pathway, in the macrophages in which inflammation has been induced by the LPS.

In one embodiment, the Stichopus japonicus-derived EV may decrease the expression of NOD-like receptor family, pyrin domain-containing 3 (NLRP3).

In one embodiment, the Stichopus japonicus-derived EV may decrease the expression of one or more selected from the group consisting of tumor necrosis factor (TNF)-α, interleukin (IL)-1β, interleukin (IL)-6, monocyte chemoattractant protein (MCP)-1, and inducible nitric oxide synthase (iNOS).

In one embodiment, the Stichopus japonicus-derived EV may increase the expression of at least one of NF-κB inhibitor alpha (IκBα) and suppressor of cytokine signaling-3 (SOCS-3).

In one embodiment, the pharmaceutical composition may decrease the level of immunoglobulin E (IgE).

In one embodiment, the pharmaceutical composition may decrease the number of mast cells in skin tissue.

In one embodiment, the pharmaceutical composition may alleviate at least one skin lesion among erythema, edema, erosion, and xerosis.

A method for preparing a pharmaceutical composition according to one embodiment of the present invention may include: decellularizing Stichopus japonicus tissue to obtain an extracellular matrix (ECM); treating the ECM with an enzyme to disrupt a structure of the ECM; and isolating and obtaining an EV from a product in which the structure of the ECM has been disrupted.

In one embodiment, the enzyme may be collagenase.

In one embodiment, the isolating and obtaining the EV may include: centrifuging the product and sequentially filtering the centrifuged product through filters to remove debris of the ECM; and ultrafiltering the product from which the debris has been removed to concentrate the EV.

In one embodiment, the removing the debris of the ECM may include sequentially centrifuging the product under conditions of 1,000×g, 5,000×g, and 10,000×g.

In one embodiment, the concentrating the EV may include concentrating the product from which the debris has been removed using a 100 kDa filter.

In one embodiment, the method of preparing the pharmaceutical composition may further include, after the ultrafiltering the product from which the debris has been removed to concentrate the EV, treating the concentrated solution with a polyethylene glycol (PEG)-based exosome extraction solution.

### BRIEF DESCRIPTION OF THE DRAWINGS

FIG. 1 is a diagram disclosing the experimental overview for confirming the therapeutic effect of Stichopus japonicus-derived extracellular vesicles (EVs) on atopic dermatitis.

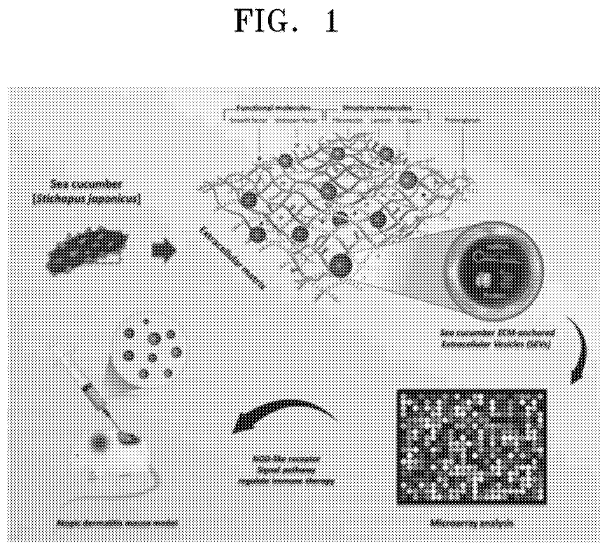

FIG. 2 is a diagram disclosing a method for extracting Stichopus japonicus-derived EVs.

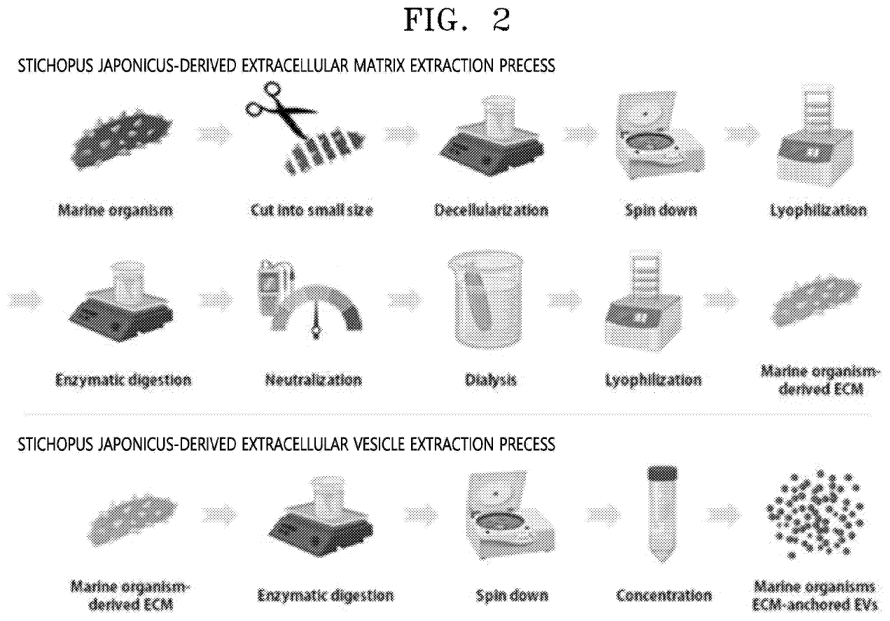

FIG. 3 is a diagram showing the results of analyzing the chemical properties of Stichopus japonicus-derived EVs.

FIG. 4 is a diagram showing the results of confirming the cytotoxicity and anti-inflammatory effects of Stichopus japonicus-derived EVs.

FIG. 5 is a diagram showing the results of confirming the nucleotide-binding oligomerization domain (NOD)-like receptor-related mechanism through large-scale gene analysis upon treatment of inflammation-induced macrophages with Stichopus japonicus-derived EVs.

FIG. 6 is a diagram showing the experimental method (a) for confirming the anti-atopic effect by subcutaneous administration of Stichopus japonicus-derived EVs in a mouse model of induced atopic dermatitis and the resulting therapeutic effects (b to h).

FIG. 7 is a diagram showing the results of confirming the anti-atopic therapeutic effects by subcutaneous administration of Stichopus japonicus-derived EVs in a mouse model of induced atopic dermatitis through histological image analysis (a and b), skin thickness measurement (c and d), and mast cell count measurement (e).

### DETAILED DESCRIPTION

Hereinafter, the present invention will be described in detail by way of examples. However, the following examples are provided as illustrations of the present invention, and when it is determined that a detailed description of a technology or feature well known to those skilled in the art may unnecessarily obscure the gist of the present invention, the detailed description may be omitted, and the present invention is not limited thereby. The present invention is capable of various modifications and applications within the scope of the claims and the equivalent scope interpreted therefrom.

In addition, the terminology used herein includes terms used to appropriately express preferred embodiments of the present invention, and may vary depending on the intention of the user or operator, or the practices of the field to which the present invention pertains. Therefore, the definitions of these terms should be determined based on the content throughout the present specification.

Unless otherwise defined, all technical terms used herein have the same meanings as commonly understood by one of ordinary skill in the art to which this invention belongs. In addition, while preferred methods and samples are described herein, similar or equivalent ones are also included within the scope of the present invention. All publications cited herein as references are incorporated into the present invention.

When a part is said to “include” a certain component in the present invention, unless specified otherwise, this means that it may further include other components, rather than exclude other components.

Stichopus japonicus is a marine invertebrate belonging to the class Holothuroidea of the phylum Echinodermata, having numerous wart-like protrusions on its dorsum. The mouth is located at the anterior end and is surrounded by numerous tentacles, while the anus is located at the posterior end. Most individuals may be distinguished as male or female, but are difficult to distinguish based on the outward appearance. When feeding, the tentacles are used to take in sand and mud from the seafloor and ingest small organisms therein, while the sand and excretions are expelled. When subjected to external stimulation, the intestine is severed and expelled through the anus, but regenerates due to strong regenerative capacity. Stichopus japonicus has long been widely used, as evidenced by the documentation of its effects on restoring vitality and stabilizing the nerves in historical texts such as Jasaneobo, Boncho Gangmok, and Dongui Bogam, and saponin components have been identified in recent studies.

Extracellular vesicles (EVs) are mediators that primarily carry functional cargo such as proteins, mRNA, microRNA, DNA, and lipids, and include “exosomes,” “microvesicles,” and “apoptotic bodies,” serving as essential elements of intercellular communication. Among EVs, exosomes and secretory microvesicles are important delivery mediators in the tumor microenvironment. EVs play an important role in intercellular communication by serving as mediators for the transfer of proteins, lipids, and RNA between the cell membrane and cytoplasm, affecting the inhibition of inflammatory responses, reduction of oxidative stress, and cell differentiation. However, there are limitations on the use of EVs due to their small quantities and difficulty in purification.

The term “nucleotide-binding oligomerization domain (NOD)-like receptor (NLR)” as used in the present invention, like retinoic acid-inducible gene (RIG)-I-like receptors and the like, refers to one of the receptors in the innate immune system that recognizes damaged cellular matrices upon attack by pathogens. The NLR is activated by pathogen-associated molecular patterns (PAMPs), damage-associated molecular patterns (DAMPs), and other harmful substances within the cell, and is a cytoplasmic protein involved in directing acquired immune responses. Among these receptors, NLR family pyrin domain-containing-10 (NLRP10), which has an unusual form lacking the leucine-rich repeat domain that binds ligands, has been extensively studied using genetically engineered NLRP10-deficient mice.

Interferon gamma inducible protein 16 (IFI16) encodes a member of the hematopoietic interferon-inducible nuclear antigen with 200 amino acid repeats (HIN-200) family of cytokines. IFI16 serves to detect intracellular DNA, which is a hallmark of virus-infected cells, and is also associated with the death of human immunodeficiency virus (HIV)-infected helper CD4 T cells via pyroptosis, which is an inflammatory disease. IFI16 is primarily expressed in response to viral infection and cellular damage, and is related to cell signaling to the immune system.

In other words, IFI16 plays a role in activating immune responses against viruses or infections in response to DNA damage in the NLR signaling pathway, but up-regulation of IFI16 in autoimmune diseases (atopic diseases) contributes to psoriasis by regulating chemokine production in keratinocytes. Therefore, in the present invention, IFI16 was used to measure the inhibition of the NLR signaling pathway.

NLR family pyrin domain-containing-3 (NLRP3), which is one of the NLRs, plays a role in regulating immune responses by detecting base sequence damage or the presence of microorganisms within the cell. NLRP3 contains a pyrin domain, which plays an important role in protein-protein interactions and signal transduction; and an NACHT domain (a nucleotide-binding and oligomerization domain in proteins containing leucine-rich repeats and pyrin domains), which is involved in ATP binding and protein multimerization and plays an important role in NLRP3 activation. Activation of NLRP3 promotes extracellular secretion of IL-1β and IL-18, which plays an important role in the regulation of inflammatory responses.

In one embodiment of the present invention, the anti-inflammatory mechanism through suppression of NLR expression was confirmed by treating inflammation-induced macrophages with Stichopus japonicus-derived EVs. In addition, the Stichopus japonicus-derived EVs were subcutaneously administered to mice in which atopic dermatitis was induced using a chemical, 2,4-dinitrochlorobenzene (DNCB), three times per week for three weeks, and the results confirmed that skin thickness, atopic severity scores, and serum immunoglobulin E (IgE) levels decreased.

The term “prevention” as used in the present invention refers to all actions that suppress or delay the progression of symptoms of a specific disease through administration of the composition of the present invention.

The term “treatment” as used in the present invention refers to all actions that improve or beneficially alter the symptoms of a specific disease through administration of the composition of the present invention.

In the pharmaceutical composition according to the present invention, the Stichopus japonicus-derived EVs may be obtained by isolating and separating an extract using extraction and separation methods known in the art. In the examples of the present invention, the extracellular matrix (ECM) of Stichopus japonicus was extracted and treated with an enzyme (collagenase) to obtain Stichopus japonicus-derived EVs.

The pharmaceutical composition may further include a pharmaceutically acceptable carrier. As used herein, the term “pharmaceutically acceptable” refers to a composition that is physiologically acceptable and does not ordinarily produce allergic reactions such as gastrointestinal disorders and dizziness, or similar reactions, when administered to humans. Examples of pharmaceutically acceptable carriers include carriers for oral administration such as lactose, starch, cellulose derivatives, magnesium stearate, and stearic acid, and carriers for parenteral administration such as water, suitable oils, saline, aqueous glucose, and glycols, and may further include stabilizers and preservatives. Suitable stabilizers include antioxidants such as sodium bisulfite, sodium sulfite, or ascorbic acid. Suitable preservatives include benzalkonium chlorides, methyl- or propyl-paraben, and chlorobutanol. Other pharmaceutically acceptable carriers are described in Remington's Pharmaceutical Sciences, 19th ed., Mack Publishing Company, Easton, PA, 1995.

The pharmaceutical composition according to the present invention may be formulated in a suitable form according to methods known in the art together with the pharmaceutically acceptable carriers described above. In other words, the pharmaceutical composition of the present invention may be prepared in various forms for parenteral or oral administration according to known methods. When formulated, diluents or excipients such as commonly used fillers, extenders, binders, wetting agents, disintegrants, and surfactants are used.

Solid preparations for oral administration may include tablets, pills, powders, granules, capsules, troches, and the like, and these solid preparations may be prepared by mixing at least one excipient, for example, starch, calcium carbonate, sucrose, lactose, gelatin, and the like, with the composition. In addition to simple excipients, lubricants such as magnesium stearate and talc may also be used. Liquid preparations for oral administration include suspensions, oral solutions, emulsions, syrups, and the like, and in addition to commonly used simple diluents such as water and liquid paraffin, various excipients such as wetting agents, sweeteners, flavoring agents, and preservatives may be included.

Preparations for parenteral administration may include sterile aqueous solutions, non-aqueous solvents, suspensions, emulsions, lyophilized preparations, and suppositories. As non-aqueous solvents and suspending agents, propylene glycol, polyethylene glycol, vegetable oils such as olive oil, and injectable esters such as ethyl oleate may be used. As a base for suppositories, Witepsol, Macrogol, Tween 61, cacao oil, laurel oil, glycerol, gelatin, or the like may be used.

The pharmaceutical composition may further include carriers, excipients, or diluents. Carriers, excipients, or diluents may include, for example, lactose, dextrose, sucrose, sorbitol, mannitol, xylitol, erythritol, maltitol, starch, acacia gum, alginate, gelatin, calcium phosphate, calcium silicate, cellulose, methyl cellulose, microcrystalline cellulose, polyvinylpyrrolidone, water, methylhydroxybenzoate, propylhydroxybenzoate, talc, magnesium stearate, or mineral oils.

The pharmaceutical composition of the present invention is administered in a pharmaceutically effective amount. The term “administration” as used in the present invention means introducing a predetermined substance to an individual by an appropriate method, and the administration route of the composition may be via any general route as long as it may reach the target tissue. Administration may be via intraperitoneal, intravenous, intramuscular, subcutaneous, intradermal, oral, topical, intranasal, intrapulmonary, intrarectal, or intracerebrovascular routes, but is not limited thereto.

The term “pharmaceutically effective amount” refers to an amount sufficient to treat a disease at a reasonable benefit/risk ratio applicable to medical treatment and not causing adverse effects. The effective dose level may be easily determined by those skilled in the art depending on factors including the patient's sex, age, body weight, health conditions, the type and severity of the disease, drug activity, sensitivity to the drug, administration method, administration time, administration route, excretion rate, treatment duration, combined and concurrent drugs, and other factors well known in the medical field.

The composition of the present invention may be used alone or in combination with surgical, hormonal, chemotherapeutic, and biological response modifier methods for the prevention or treatment of atopic dermatitis.

The term “amelioration” as used in the present invention refers to all actions that at least reduce a parameter associated with the condition being treated, for example, the severity of a symptom.

The food composition of the present invention may include health functional foods. The term “health functional food” as used in the present invention refers to a food manufactured and processed in the form of tablets, capsules, powders, granules, liquids, and pills using raw materials or ingredients with functionalities useful to the human body. Here, the term “functionality” refers to regulating nutrients for the structure and function of the human body or obtaining a useful effect for health purposes such as physiological effects. The health functional food of the present invention may be manufactured by a method commonly used in the art, and may be manufactured by adding raw materials and ingredients commonly added in the art during the manufacturing process. In addition, the health functional food may be manufactured into any formulation without limitation as long as it is a formulation recognized as a health functional food. The food composition of the present invention may be manufactured in various forms of formulations, and unlike general drugs, it has the advantage of having no side effects that may occur when taking drugs for a long time since a food is used as a raw material, and has excellent portability. Therefore, the health functional food of the present invention may be consumed as a supplement to enhance the effect of preventing or ameliorating atopic dermatitis.

The health food and health functional food compositions of the present invention contain Stichopus japonicus-derived EVs according to the present invention as essential ingredients in the indicated proportions, and there are no particular restrictions on other ingredients. Like in ordinary beverages, various flavoring agents or natural carbohydrates may be contained as additional ingredients. Examples of the above-described natural carbohydrates include monosaccharides such as glucose and fructose; disaccharides such as maltose and sucrose; polysaccharides such as conventional sugars like dextrin and cyclodextrin; and sugar alcohols such as xylitol, sorbitol, and erythritol. As flavoring agents other than those described above, natural flavoring agents (thaumatin, stevia extract (e.g., rebaudioside A, glycyrrhizin, etc.)) and synthetic flavoring agents (saccharin, aspartame, etc.) may be advantageously used.

In addition to the above, the health food and health functional food compositions including Stichopus japonicus-derived EVs according to the present invention may contain various nutrients, vitamins, minerals (electrolytes), flavoring agents such as synthetic and natural flavoring agents, coloring agents and enhancers (cheese, chocolate, etc.), pectic acid and salts thereof, alginic acid and salts thereof, organic acids, protective colloid thickeners, pH adjusters, stabilizers, preservatives, glycerin, alcohols, carbonating agents used in carbonated beverages, and the like. In addition, the health food and health functional food compositions of the present invention may contain pulp for the production of natural fruit juices, fruit juice beverages, and vegetable beverages.

These ingredients may be used independently or in combination. The proportion of such additives is not so critical, but is generally selected in the range of 0.1 to about 20 parts by weight based on 100 parts by weight of the health food and health functional food composition containing the active substance of the present invention, but is not limited thereto.

In addition, there are no restrictions on the type of health food in which the composition of the present invention can be used. Furthermore, the composition including Stichopus japonicus-derived EVs of the present invention as an active ingredient may be prepared by mixing it with other appropriate auxiliary ingredients and known additives that may be contained in health functional foods at the discretion of those skilled in the art. Examples of foods that may be added include meats, sausages, breads, chocolates, candies, snacks, confectioneries, pizzas, ramen noodles, other noodles, gums, dairy products including ice cream, various soups, beverages, teas, drinks, alcoholic beverages, and vitamin complexes, and the food may be prepared by adding it to juices, teas, jellies, and juices prepared with the extract of the present invention as a main ingredient.

Hereinafter, the present invention will be described in more detail with reference to the following examples. However, the following examples are merely illustrative of the present invention, and the content of the present invention is not limited by the following examples.

### <Example 1> Preparation of Stichopus japonicus-Derived Extracellular Vesicles (EVs)

Stichopus japonicus was purchased from a fish market, and foreign matter was removed using fresh water, after which the viscera were removed and the Stichopus japonicus was stored frozen. The frozen Stichopus japonicus (100 to 200 g) was cut into small pieces, placed in a storage solution, and stirred at 4 °C for three days to perform decellularization. Thereafter, centrifugation was performed at 10,000×g for 10 minutes, and the extracellular matrix (ECM) of the Stichopus japonicus precipitated at the bottom was lyophilized. The lyophilized Stichopus japonicus ECM was allowed to react with collagenase for 48 hours to disrupt the structural framework, and then the resulting product was subjected to sequential centrifugation (1,000×g, 5,000×g, and 10,000×g) to remove relatively larger debris. Thereafter, ECM debris was removed using 450 nm and 200 nm filters, and EVs were concentrated using a 100 kDa filter. The concentrated Stichopus japonicus EV solution was repeatedly diluted and concentrated using saline, and soluble proteins were removed during the process. Finally, Stichopus japonicus-derived EVs were obtained using a polyethylene glycol (PEG)-based exosome extraction solution.

### <example 2> Characterization of Stichopus Japonicus-Derived EVs

For the Stichopus japonicus-derived EVs of the present invention, scanning electron microscopy (SEM), transmission electron microscopy (TEM), dynamic light scattering (DLS), uptake analysis, and protein and gene electrophoresis of the internal contents of the Stichopus japonicus-derived EVs were performed.

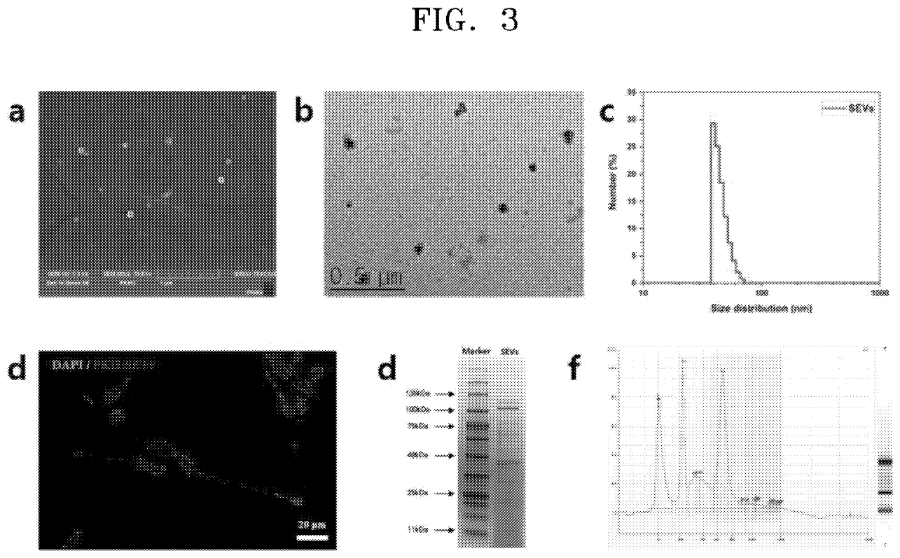

Through this, as shown in FIG. 3, the morphological results of the electron microscopy images showed a spherical or circular shape, and the size distribution determined by DLS analysis ranged from 29.38019 nm to 71.41572 nm. RAW-264.7 cells were treated with Stichopus japonicus-derived EVs labeled with PKH-26, confirming that endocytosis was successfully observed without stimulation.

To confirm the components of the Stichopus japonicus-derived EVs, proteins and genetic materials were analyzed. Protein electrophoresis confirmed the presence of various proteins in the Stichopus japonicus-derived EVs, including structural proteins. Gene electrophoresis of internal genetic material confirmed the presence of large amounts of 22 nt miRNA, 67 nt tRNA, small rRNA, and the like.

### <Example 3> Confirmation of Cytotoxicity of Stichopus japonicus-Derived EVs

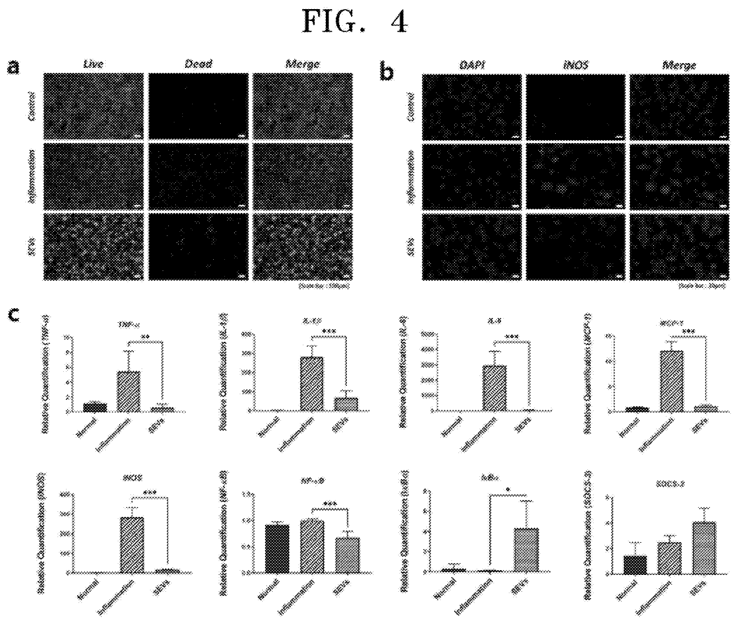

To confirm the cytotoxicity of the Stichopus japonicus-derived EVs of the present invention, macrophages were exposed to 10 μg/mL of the EVs of Example 1 and lipopolysaccharide (LPS) at 50 ng/mL. Thereafter, cell viability was assessed, and as shown in FIGS. 4A and 4B, it was confirmed that cell death in the Stichopus japonicus-derived EV-treated group did not substantially increase compared to the control group and the inflammation-induced group. Accordingly, experiments in the following examples were performed using 10 μg/mL of the Stichopus japonicus-derived EVs.

### <Example 4> Confirmation of Anti-Inflammatory Effects of Stichopus japonicus-Derived EVs

To confirm the anti-inflammatory effects of the EVs of Example 1, real-time PCR and immunocytochemistry experiments were performed. Macrophages were cultured in a 24-well plate, and after 24 hours, the cells were treated with a Stichopus japonicus-derived EV suspension (10 μg/mL) for 48 hours in Roswell Park Memorial Institute (RPMI)-1640 medium without fetal bovine serum (FBS) containing LPS (50 ng/mL).

A spin column extraction kit was used for RNA extraction, and the RNA was then transcribed using a cDNA synthesis kit. Quantitative real-time PCR was performed using QuantStudio1 containing 2X SYBR Green Reaction Mix and specific primers, and the relative gene expression levels were normalized using the endogenous control glyceraldehyde 3-phosphate dehydrogenase (GAPDH).

Through this, as shown in FIG. 4c, the expression levels of pro-inflammatory cytokines and inflammatory factors were compared, and the results confirmed that the group treated with the Stichopus japonicus-derived EVs had a statistically significant inhibitory effect compared to the inflammation group. Tumor necrosis factor (TNF)-α decreased by 89.04% (p=0.005), interleukin (IL)-1β by 75.41% (p<0.001), IL-6 by 99.19% (p<0.001), monocyte chemoattractant protein (MCP)-1 by 90.05% (p<0.001), inducible nitric oxide synthase (iNOS) by 93.97% (p<0.001), and nuclear factor kappa-light-chain-enhancer of activated B cells (NF-κB) by 33% (p<0.001). In addition, it was confirmed that the expression levels of anti-inflammatory inhibitory factors increased. The NF-κB inhibitor alpha (IκBα), which is an inhibitor of NF-κB activation, increased by 3,048% (p=0.011), and suppressor of cytokine signaling (SOCS)-3 increased by 61.7% (p=0.155).

In addition, iNOS protein expression was confirmed through immunofluorescence analysis for 48 hours after treatment with the Stichopus japonicus-derived EVs, and the results confirmed that the protein expression (fluorescence expression level) was decreased in the group treated with the Stichopus japonicus-derived EVs.

### <Example 5> Confirmation of Anti-Atopic Mechanism of Stichopus japonicus-Derived EVs Through Gene Analysis

To confirm the anti-inflammatory effect mechanism of the Stichopus japonicus-derived EVs of Example 1, large-scale gene analysis (microarray) was performed.

Macrophages were cultured in a 24-well plate, and after 24 hours, the cells were treated with a Stichopus japonicus-derived extracellular vesicle suspension (10 μg/mL) for 48 hours in RPMI-1640 medium without FBS containing LPS (50 ng/mL).

A spin column extraction kit was used for RNA extraction, and the purified RNA was sent to Macrogen (Seoul, Korea) for large-scale gene analysis.

For gene expression analysis and statistical analysis, data was summarized and normalized using the signal space transformation-robust multichip analysis (SST-RMA) method implemented in Affymetrix® Power Tools (APT). The results were exported from gene-level SST-RMA analysis and differentially expressed gene (DEG) analysis was performed. Statistical significance of the expression data was determined using the local-pooled-error (LPE) test applying the null hypothesis of no difference between groups and fold change, and p-values were adjusted using the Benjamini-Hochberg algorithm to control the false discovery rate (FDR). Hierarchical cluster analysis of the DEG sets was performed using complete linkage and Euclidean distance as measures of similarity. Gene-enrichment and functional annotation analysis for the significant probe list was performed using Gene Ontology (GO) (http://geneontology.org) and Kyoto Encyclopedia of Genes and Genomes (KEGG) (http://kegg.jp). All data analysis and visualization of the DEGs were performed using R 3.3.2.

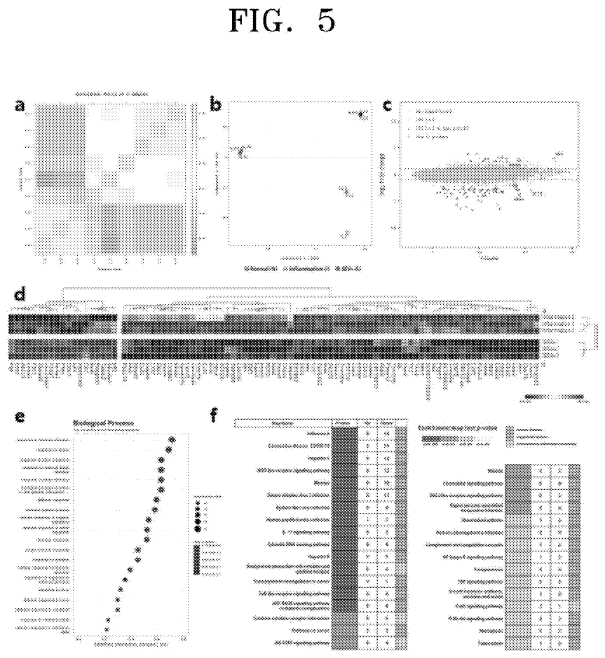

Through this process, as shown in FIG. 5, a Pearson's correlation test and multidimensional scaling showed the overall changes in gene patterns of each group. The correlation analysis results showed changes in gene expression patterns, as the correlation values decreased in the group treated with the Stichopus japonicus-derived EVs (N_I vs N_S: 0.977±0.005, N_I vs N_I: 0.990±0.009, N_S vs N_S: 0.994±0.004).

The expression of 22,206 genes was compared between the inflammation group and the EV group, and hierarchical clustering identified 92 genes with statistically significant differences. In particular, the transcription levels of 73 target genes were confirmed to be down-regulated and 19 genes to be up-regulated in response to EVs. In addition, the top 5 ranked genes satisfying statistical significance were marked with red dots. Three types of down-regulated genes were identified: C-C motif chemokine ligand 4 (CCL4), schlafen 4 (Slfn4), and interferon γ inducible protein 16 (IFI16); and two types of up-regulated genes: metallothionein 2 (MT2) and ATP-binding cassette subfamily C member 5 (ABCC5).

In the GO analysis, responses to external stimuli, stress, other organisms, biological stimuli, biological processes related to interspecies interactions between organisms, defense responses, immune system processes, cellular responses to organic substances, defense responses to other organisms, immune responses, innate immune responses, responses to cytokines, cellular responses to cytokine stimuli, regulation of responses to external stimuli, responses to viruses, defense responses to symbionts, responses to interferon-beta, and cellular responses to interferon-beta were observed in the group treated with Stichopus japonicus-derived EVs.

The KEGG analysis confirmed that Stichopus japonicus-derived EVs are involved in the NLR signaling pathway, IL-17 signaling pathway, cytosolic DNA sensing pathway, viral protein interaction with cytokine and cytokine receptor, Toll-like receptor signaling pathway, cytokine-cytokine receptor interaction, Janus kinase-signal transducer and activator of transcription (JAK-STAT) signaling pathway, chemokine signaling pathway, retinoic acid-inducible gene (RIG)-I-like receptor signaling pathway, NF-κB signaling pathway, TNF signaling pathway, growth hormone synthesis, Forkhead box O (FoxO) signaling pathway, and phosphoinositide 3-kinase-protein kinase B (PI3K-Akt) signaling pathway.

Accordingly, it was confirmed that the pathogenic mechanism of atopic disease among diseases involving NLR has a high relevance to the physiological activity of Stichopus japonicus-derived EVs, and the application of Stichopus japonicus-derived EVs was expanded to an atopic model.

### <Example 6> Confirmation of Anti-Atopic Effects of Stichopus japonicus-Derived EVs in Non-Human Animals

The experiments in Example 6 were performed using animal protocols approved by the Pukyong National University Institutional Animal Care and Use Committee (PKNUIACUC-2023-05).

### <6.1> Measurement of Skin Severity Score

Atopic dermatitis was induced in 4-week-old male BALB/c mice purchased from SAMTACO BIO KOREA CO., LTD. 2,4-Dinitrochlorobenzene (DNCB) was dissolved in acetone-olive oil (AOO) at a ratio of 4:1, with AOO used as the vehicle.

The dorsal skin was shaved using an electric clipper and a depilatory cream. At 10 days and 6 days before the start of the experiment, the dorsal skin and ears were treated with 150 μL of 1% DNCB solution to sensitize the mice. Subsequently, a 0.2% DNCB solution was applied at 2 days and 0 days before (on the day of the experiment). A test was conducted by repeatedly applying 150 μL of 0.2% DNCB solution to the mice three times per week for three weeks, and 200 μL of Stichopus japonicus-derived EVs (1 or 10 μg/mL) was subcutaneously injected during the same period.

Regarding the progression of atopic dermatitis, the severity of dermatitis was clinically assessed using methods established in previous studies, as described below.

The total dermatitis severity score was calculated as the sum of individual scores for each of the four signs and symptoms of erythema/bleeding, edema, scaling/erosion, and dryness (0: asymptomatic, 1: mild, 2: moderate, 3: severe). The individual scores were summed to determine the overall dermatitis score (maximum score: 12).

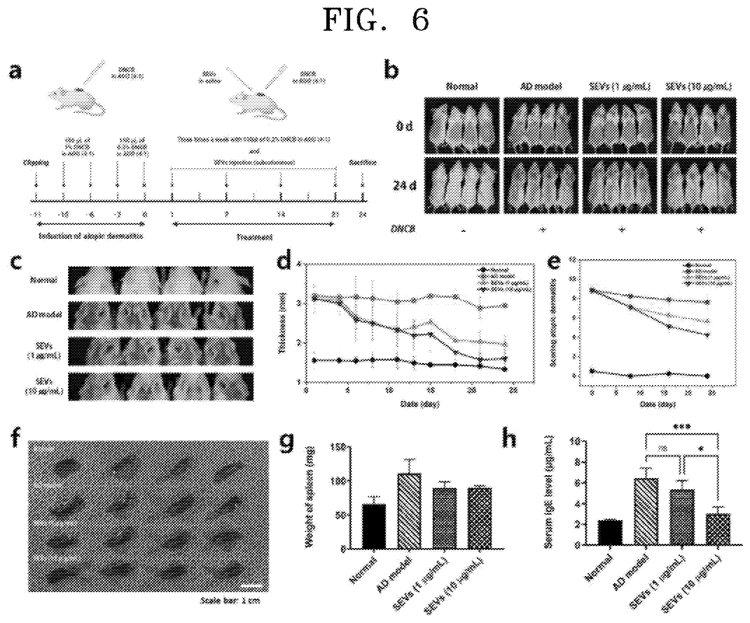

Through this, as shown in FIG. 6e, it was confirmed that the clinical dermatitis assessment scores in the groups treated with the Stichopus japonicus-derived EVs were significantly decreased, and that the dermatitis assessment score in the group treated with high-concentration Stichopus japonicus-derived EVs was further decreased (normal: 0±0, AD model: 7.63±0.75, EVs (1 μg/mL): 5.63±0.48, EVs (10 μg/mL): 4.25±0.50).

### <6.2> Measurement of Immunoglobulin E Levels

First, as a characteristic of chronic inflammatory skin disease, the spleen weight was measured to confirm whether hypertrophy of immune organs such as the spleen was present.

Through this, as shown in FIG. 6g, it was confirmed that the spleen weight of the normal model was 65.87 mg±11.19, and that of the atopic model was 111.00 mg±20.28, whereas the spleen weight of the group treated with the Stichopus japonicus-derived EVs in an amount of 1 μg/mL was 89.90 mg±8.94, and that of the group treated with the Stichopus japonicus-derived EVs in an amount of 10 μg/mL was 90.07 mg±3.08.

Thereafter, regarding immunoglobulin E (IgE) levels, mice were anesthetized with isoflurane, and blood samples were collected. Serum was separated by centrifugation and stored at −80 °C until analysis, and IgE levels were measured using an enzyme-linked immunosorbent assay kit according to the manufacturer's instructions.

Through this, as shown in FIG. 6h, it was confirmed that while the atopic dermatitis mouse model exhibited increased IgE levels in the serum, the mouse model group treated with the Stichopus japonicus-derived EVs exhibited a significant decrease in IgE levels. Specifically, it was confirmed that the IgE release levels in the groups treated with low-concentration and high-concentration Stichopus japonicus-derived EVs were inhibited to approximately 83±13.1% and 47±10.1%, respectively.

### <6.3> Histological Image Analysis

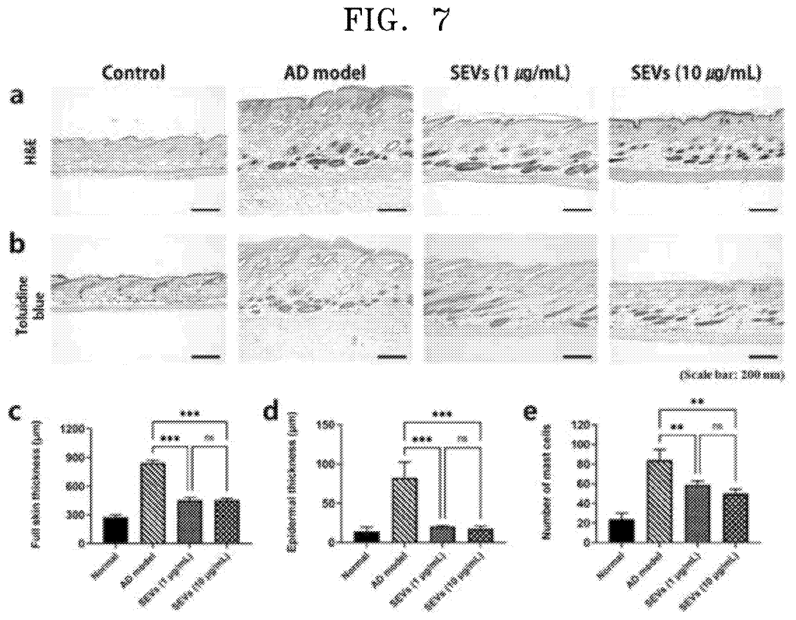

To analyze histological images, dorsal skin samples were fixed in 10% formaldehyde and embedded in paraffin, and as shown in FIGS. 7A and 7B, toluidine blue staining and hematoxylin and eosin (H&E) staining were used to observe epidermal proliferation and immune cell infiltration in the dermis. Image J software was used to measure thickness, and five fields were randomly selected to count the number of mast cells.

Thereafter, the skin thickness of the mouse models was measured using vernier calipers, and through this, as shown in FIG. 7d, it was confirmed that skin thickness in the groups treated with the Stichopus japonicus-derived EVs was statistically decreased, and that the increase in skin thickness was further suppressed in the high-concentration group (normal: 1.34 mm±0.06, atopic model: 2.95 mm±0.04, Stichopus japonicus-derived EVs (1 μg/mL): 1.96 mm±0.22, and Stichopus japonicus-derived EVs (10 μg/mL): 1.60 mm±0.27).

In addition, as shown in FIG. 7e, it was confirmed that the number of mast cells was statistically decreased in the groups treated with the Stichopus japonicus-derived EVs (normal: 24.5±5.5, atopic model: 86.2±10.8, Stichopus japonicus-derived EVs (1 μg/mL): 56.4±6.5, and Stichopus japonicus-derived EVs (10 μg/mL): 44.3±6.1).

### <6.4> Confirmation of Amelioration of Skin Lesions

Amelioration of atopic dermatitis-related skin lesions in the groups treated with the Stichopus japonicus-derived EVs was confirmed visually.

Through this, as shown in FIG. 6b, while the dorsal skin of the atopic dermatitis mouse model exhibited pronounced erythema, edema, scaling, and dryness, it was confirmed that these symptoms were alleviated in the groups treated with the Stichopus japonicus-derived EVs. In addition, as shown in FIG. 6c, observation of the ear lesion areas confirmed that erythema and scaling were alleviated by application of the Stichopus japonicus-derived EVs.

Furthermore, regarding the symptoms of skin lesions, the symptoms of erythema, edema, erosion, and dryness of the skin lesions were exacerbated in the control group compared to the normal group, whereas the symptoms were ameliorated in the groups administered the Stichopus japonicus-derived EVs compared to the control group.

The preferred embodiments of the present invention have been described above. Those skilled in the art to which the present invention pertains will understand that the present invention can be implemented in a modified form without departing from the essential characteristics of the present invention. Therefore, the disclosed embodiments should be considered from an illustrative perspective rather than a limiting perspective. The scope of the present invention is indicated particularly in the claims, not in the foregoing description, and all differences within the equivalent scope shall be construed as being included in the present invention.

The composition including the Stichopus japonicus-derived extracellular vesicles (EVs) of the present invention has an excellent therapeutic effect on atopic dermatitis, and can be utilized in the field of researching better therapeutic effects, and thus can be used in useful businesses related to atopic dermatitis.

What is claimed is:

1. A pharmaceutical composition for preventing and treating atopic dermatitis, comprising a Stichopus japonicus-derived extracellular vesicle (EV) as an active ingredient.

2. The pharmaceutical composition of claim 1, wherein the Stichopus japonicus-derived EV is obtained by enzymatically treating an extracellular matrix (ECM) obtained by decellularizing Stichopus japonicus tissue.

3. The pharmaceutical composition of claim 2, wherein the enzyme is collagenase.

4. The pharmaceutical composition of claim 1, wherein the Stichopus japonicus-derived EV includes an exosome.

5. The pharmaceutical composition of claim 1, wherein the Stichopus japonicus-derived EV suppresses an inflammatory response by participating in a nucleotide-binding oligomerization domain (NOD)-like receptor signaling pathway in macrophages in which inflammation has been induced by lipopolysaccharide (LPS).

6. The pharmaceutical composition of claim 5, wherein the Stichopus japonicus-derived EV decreases the expression of interferon gamma inducible protein 16 (IFI16), a gene associated with the NOD-like receptor signaling pathway, in the macrophages in which inflammation has been induced by the LPS.

7. The pharmaceutical composition of claim 1, wherein the Stichopus japonicus-derived EV decreases the expression of NOD-like receptor family, pyrin domain-containing 3 (NLRP3).

8. The pharmaceutical composition of claim 1, wherein the Stichopus japonicus-derived EV decreases the expression of one or more selected from the group consisting of tumor necrosis factor (TNF)-α, interleukin (IL)-1β, interleukin (IL)-6, monocyte chemoattractant protein (MCP)-1, and inducible nitric oxide synthase (iNOS).

9. The pharmaceutical composition of claim 1, wherein the Stichopus japonicus-derived EV increases the expression of at least one of NF-κB inhibitor alpha (IκBα) and suppressor of cytokine signaling-3 (SOCS-3).

10. The pharmaceutical composition of claim 1, wherein the pharmaceutical composition decreases the level of immunoglobulin E (IgE).

11. The pharmaceutical composition of claim 1, wherein the pharmaceutical composition decreases the number of mast cells in skin tissue.

12. The pharmaceutical composition of claim 1, wherein the pharmaceutical composition alleviates at least one skin lesion among erythema, edema, erosion, and xerosis.

13. A method for preparing a pharmaceutical composition, comprising:
decellularizing Stichopus japonicus tissue to obtain an extracellular matrix (ECM);
treating the ECM with an enzyme to disrupt a structure of the ECM; and
isolating and obtaining an EV from a product in which the structure of the ECM has been disrupted.

14. The method of claim 13, wherein the enzyme is collagenase.

15. The method of claim 13, wherein the isolating and obtaining the EV includes:
centrifuging the product and sequentially filtering the centrifuged product through filters to remove debris of the ECM; and
ultrafiltering the product from which the debris has been removed to concentrate the EV.

16. The method of claim 15, wherein the removing the debris of the ECM includes sequentially centrifuging the product under conditions of 1,000×g, 5,000×g, and 10,000×g.

17. The method of claim 15, wherein the concentrating the EV includes concentrating the product from which the debris has been removed using a 100 kDa filter.

18. The method of claim 15, further comprising, after the ultrafiltering the product from which the debris has been removed to concentrate the EV, treating the concentrated solution with a polyethylene glycol (PEG)-based exosome extraction solution.
